(19) United States
(12) Patent Application Publication
(10) Pub. No.: US 2024/0245744 A1
(43) Pub. Date: Jul. 25, 2024

(54) INTRANASAL ADMINSTRATION OF CANNABIGEROL, CBDa, CBGa, & UNCARIA TOMENTOSA HYDROALCOHOLIC EXTRACT COMPOUNDS EMULSIFIED IN SALINE HOMOGENOUS SOLUTION FOR INTRANASAL ADMINISTRATION

(71) Applicant: Artis Asahel Clark, Ann Arbor, MI (US)

(72) Inventor: Artis Asahel Clark, Ann Arbor, MI (US)

(21) Appl. No.: 18/099,943

(22) Filed: Jan. 22, 2023

(51) Int. Cl. A61K 36/74; A61K 9/00; A61K 9/107; A61K 31/00

(52) U.S. Cl. CPC A61K 36/74; A61K 9/0043; A61K 9/107; A61K 31/658

(57) ABSTRACT

The therapeutic product invention states to the use of quantified solutions containing natural compounds of cannabidiol, CBDa, cannabigerol, CBGa combined with Uncaria tomentosas' hydroalcoholic extracts emulsified in saline solution in homogenous mixture administered intranasally. A natural respiratory immune booster with mechanisms against respiratory viruses and foreign substances (e.g. SARS-Cov-2 spike protein, bacterial, allergen) including; inhibition, reduction, blockage of cellular entry, and encapsulation and expulsion with minute impact of variant lineage. By induction of the interferon pathway both directly and indirectly following activation of the host immune response to viral pathogen, suppressing cytokine activation in response to viral infection, as well as blocks viral replication after entry into cells. Attaching to viral protein, mechanically inhibiting the SARS-COV-2 enzyme 3CLpro and disrupts the interface of the receptor-binding domain of angiotensin-converting enzyme 2 (RBD-ACE-2) as well as the SARS-COV-2 spike glycoprotein.

### FIELD

The present application relates to a homogenous solution of natural formulation, specifically utilizing cannabidiol, CBDa, cannabigerol, CBGa and Uncaria Tomentosa solute emulsified in saline solvent for intranasal administration.

### BACKGROUND

Nasal spray products are used to target the respiratory system that is the main entry point for respiratory benefits, hygienics, and relief. Respiratory illnesses, viruses, bacteria, and allergens are often a product of an individual's health, surroundings or public emergencies that can be of detriment to a vast representation of subjects in society. There are a small number of natural health options coupled with the host immune system, drugs or pharmaceuticals are the most prevalent option of defense against these ailments, the latter often presented with various side effects. Public sentiment for all natural herbal remedies or therapeutics have become popular as prevention or defense against these detriments upon a subject's health.

### SUMMARY

By providing a product that is of all-natural make in homogenous solution and delivered intranasally the risk of side-effects is reduced and public trust is gained by alternative safety provided.

The produced exemplary embodies a product that is fully all-natural with organic elements.

In one instance the formulation takes advantage of 100% of the solute possible to apply in solution for maximum effect of product mixture.

Another representation of product takes advantage of 75% of the solute applicable within solution to keep effect and add additional relief.

Other embodiments provide for increased relief with similar dilutions of solute made.

The additional representations, features, advantages, and properties of the product in the current application will be further recognized within the detailed description.

### BRIEF DESCRIPTION OF THE DRAWINGS

In the following detailed portion of the present description, the instruction of the current application will be described in further detail with reference to the example manifestation shown in the illustrations, in which:

FIG. 1 is a representational view of the product and its four diluted and less concentrated embodiments at 100%, 75%, 50%, and 25%.

### DETAILED DESCRIPTION

The following detailed description, the product pertaining to the instructions for this application in the form of a nasal spray will be described by the representations. It should be noted that although only a nasal spray is explained the instructions of this application can also be used in any other intranasal irrigation device such as a nasal douche, nasal drops and other product devices that offer irrigation of nostrils, nasal passage, and throat larynx.

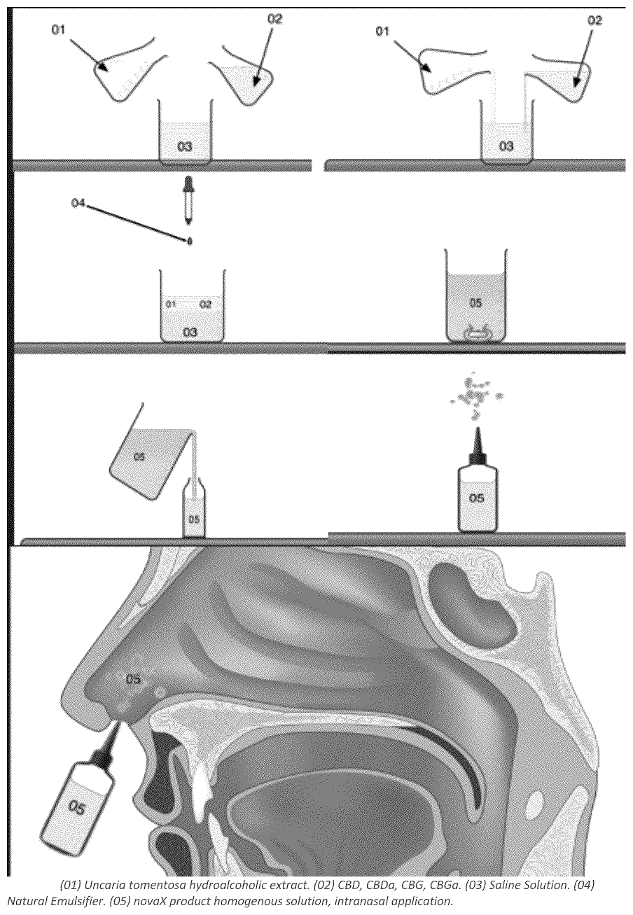

The first representation of the all-natural homogenous solution product is displayed in FIG. 1. The compounds that make up the solute at 100% are (01) representing Uncaria tomentosa hydroalcoholic extract constituents in the amount of 8.6 mL containing 7,678.57 mg of Uncaria tomentosa giving an approximate concentration of 0.89 ug/uL in organic vegetable glycerin and distilled water. Item (02) is the cannabidiol, CBDa, cannabigerol and CBGa extract quantities in separate mixture of themselves with each cannabinoid compound individual amount at 90.1 mg giving a total amount of cannabinoids (cannabidiol, CBDa, cannabigerol, CBGa) of 360.4 mg within the 5.3 mL added into product mixture, in total giving a concentration of cannabinoids added at 0.068 mg/uL in fractionated organic coconut oil and full spectrum hemp oil.

In this embodiment both (01) and (02) are added to (03) the saline solution of amounts 21.6 mL consisting of a normal formulation of 0.65-0.9% sodium chloride (salt) within purified water with added ingredients of Disodium Phoshate, Phenylcarbinol, Monosodium Phosphate, and Benzalkonium Chloride. The dropwise addition of (04) is the emulsifying agent in the amount of 1.06 mL (pertaining to liquid sunflower lecithin or alternative all-natural emulsifier if needed). These amounts are mixed by magnetic stir bar to create the homogenous product solution of (05) that is dispersed in singularity intranasally to both nostrils with addition to throat larynx.

In the present embodiment the homogenous mixture produced (05) is the final product novaX which is measured as needed per housing container size. In the illustration a 30.0 mL nasal spray bottle of eco-friendly material is represented in container and dispersing (05) mist within nostrils or wash depending on device.

In this representation it is noted that (05) can be mixed into large batches of homogenous solutions and distributed into higher or lower amounts of different types of intranasal devices in allocation in minor or mass production manufacturing operations of the same concentration of the current embodiment of 100% of the solute created of the components (01), (02), (03), and (04) concentrations in unison to scale of the bulk production solution intended to create and distribute. Additionally, the order of the (01), (02), (03), and (04) components are added in can be in any order as long as they still form a homogenous solution product (05) containing scaled amounts of (01), (02), (03), and (04) concentrations pertaining to the final product (05) amount desired.

The apparatus's used are not described in detail due to the technology and all device administration of product possible is widespread and well known. The directed application is strictly for the (05) product produced from the mentioned components.

The second embodiment is traditionally described with FIG. 1 but detailed differently at 75% solute ratio to the aforementioned 100% solute ratio and items included. This is described as the content ratios of the solute are regulated to 75% of their original state producing amounts of (01) at 6.45 mL and (02) 4.0 mL introduced into adjusted 19.55 mL (03) solution with emulsifying agent (04) kept within the same 0.795-1.06 mL amount to keep homogenous solution. With the same noted elements of the first written representation elements regarding mixing order and larger or smaller production scales.

The third embodiment is also traditionally described with FIG. 1 but detailed differently at 50% solute ratio to the aforementioned 100% solute ratio and items included. This is described as the content ratios of the solute are regulated to 50% of their original state producing amounts of (01) at 4.3 mL and (02) 2.65 mL introduced into adjusted 23.05 mL (03) solution with emulsifying agent (04) kept within the same 0.50-1.06 mL amount to keep homogenous solution. With the same noted elements of the first written representation elements regarding mixing order and larger or smaller production scales.

The fourth embodiment is still traditionally described with FIG. 1 but detailed differently at 25% solute ratio to the aforementioned 100% solute ratio and items included. This is described as the content ratios of the solute are regulated to 25% of their original state producing amounts of (01) at 2.15 mL and (02) 1.33 mL introduced into adjusted 26.52 mL (03) solution with emulsifying agent (04) kept within the same 0.27-1.06 mL amount to keep homogenous solution. With the same noted elements of the first written representation elements regarding mixing order and larger or smaller production scales.

The four different embodiments described above, and their components can be used alone or in various combinations. It is still noted that these different concentrations can also be used with different intranasal devices and applied with scale to different therapies.

The instruction of the present application allows for multiple advantages in all-natural therapeutics and therapies. Different representations or implementations can possibly produce several or more of the following advantages. It is noted that though quite a large list and there are different advantages that are not described within. One example is that this product to be transferred along the nasopharyngeal factory cells human airway epithelial cells, bypassing the whole body and entering the lungs directly, which is advantageous in the treatment of respiratory symptoms associated with patients suffering from respiratory illnesses, viruses, bacteria or allergens for example SARS-Cov-19 and similar respiratory issues. An additional example is that the present invention relates to the use of all-natural therapeutic formulations delivered intranasally is a direct Central Nervous System (CNS) delivery of all-natural therapeutic formulations containing hydroalcoholic, cannabidiol, CBDa, cannabigerol, and CBGa extract compounds without invasiveness or major complications. In addition, it causes rapid increases in CNS levels of these cannabinoid and hydroalcoholic extract compounds. Intranasal administration represents the most promising, novel, non-invasive method for delivering therapeutic formulations containing cannabinoids and hydroalcoholic extract compounds directly to the CNS in the treatment of ailments for example respiratory viruses like SARS-Cov-2 particularly when used as a prevention or treatment depending on severity of extended use needed due to respiratory virus symptoms.

Though the instruction of the present application has been explained in detail for the purpose of illustration, it is to understand that the detail is mainly for that motive, and variations can be made therein by those skilled in the chemistry without separating the scope of the instruction of this application. The example at hand would be explained by the concentrations of the solute compounds (01) and (02) in regard to the emulsifying agent (04) and the major solvent (03), it can be interpreted that the mixture concentrations could be altered in any amount to help in the therapeutic effects of the all-natural plant extracts used.

Another example, although the instruction of the current application has been detailed for the use of an intranasal device, it can be appreciated that the teachings of the present application can possibly be applied to other types of therapeutic ways of administration such as within a pill or gummy vitamin taken oral for example. It is also noted that there are many alternatives to implementing the methods of the instruments and methods of the instructions in the current application.

Features described in the preceding description may be used in combinations other than the combinations explicitly described.

Whilst endeavoring in the foregoing specification to draw attention to those features of the invention believed to be of particular importance it should be understood that the Applicant claims protection in respect of any patentable feature or combination of features hereinbefore referred to and/or shown in the drawings whether or not particular emphasis has been placed thereon.

The term ‘comprising” as used in the claims does not exclude other elements or steps. The term “a” or “an” as used in the claims does not exclude a plurality. A unit or other means may fulfill the functions of several units or means recited in the claims.

1. The present invention relates to the use of therapeutic product formulations containing components of cannabidiol, CBDa, cannabigerol, CBGa and uncaria tomentosa hydroalcoholic extract compounds in solution delivered intranasally for the reduction of total viral load and spread frequency in the treatment of “treatment-resistant viruses”, for administration via a pump action nasal, a nasal douche, nasal drops or intranasal irrigation device through the respiratory system as well as crossing the blood-brain barrier via the olfactory system, to increase the availability of medications for treatment respiratory viruses for example SARS-Cov-2 or other respiratory: allergens, bacteria, illnesses or foreign substances.

2. The therapeutic product formulations as claimed in claim 1, which is packaged for delivery as a pump-action nasal spray and delivered via nasal spray or other intranasal irrigation device.

3. In accordance with claim 1, and administration in particular, the invention relates to all-natural product formulations, for use in the administration of therapeutics or therapy intranasally via respiratory system as well as olfactory transfer, comprising at least one formulation containing these certain components named within claim.

4. In accordance with claim 1, therapy functions by induction of the interferon pathway both directly and indirectly following activation of the host immune response to viral pathogen, suppressing cytokine activation in response to viral infection, as well as blocks viral replication after entry into cells.

5. A therapeutic product formulation in accordance with claim 1, comprised of essentially one or more cannabinoids and uncaria tomentosa constituents in particular cannabidiol, CBDa, cannabigerol, CBGa and spirooxindole alkaloids, indole glycoside alkaloids, quinovic acid glycosides, and proanthocyanidins.

6. In accordance with claim 1, in product delivered intranasally, through respiratory system as well as crossing the blood-brain barrier.

7. A therapeutic product formulation consisting essentially of one or more Cannabinoids and rubiaceae hydroalcoholic extract compounds.

8. A therapeutic product comprised essentially of one or more cannabinoids and Uncaria tomentosa (Rubiaceae) hydroalcoholic extract compound constituents in saline solution in homogenous solution by all-natural oil-water emulsifier for example liquid sunflower lecithin.

9. A product in accordance to claim 7, whereas the product has 1 to 2 constituents that have antiviral, inhibition, or blockage therapeutic effects.

10. In accordance with claim 7, a therapeutic product with extract constituents characterized as speciophylline, isopteropodine, isomitraphylline, uncarine F, mitraphylline, pteropodine, rhynchophylline, iso-rynchophylline, 3-dihyrocadambine, proanthocyanidin C1, epi-afzelechin-4B-8, proanthocyanidin B2, epicatechin, and chlorogenic acid, and protonated quasimolecular ions of isomeric quinovic acid glycosides for perspective constituents are important as well in the case of this invention.

11. In accordance with claim 8, a therapeutic product over or under the counter formulation consisting essentially of one or more cannabinoids and rubiaceae hydroalcoholic extract compounds in saline solution emulsified by sunflower lecithin extract.

12. In accordance with claim 8, mechanically inhibiting the virus for example SARS-COV-2 enzyme 3CLpro and disrupts the interface of the receptor-binding domain of angiotensin-converting enzyme 2 (RBD-ACE-2) as well as the SARS-COV-2 spike glycoprotein, respectively.

13. In accordance with claim 8, this therapeutic reaction halts the virus from attaching to the host cell, assisting with reduction, and exuding viral load and transmission of COVID-19.

14. Comprising with claim 8, a therapeutic product comprised essentially of one or more Uncaria tomentosa (or rubiceaes) and vegetable glycerin solute.

15. Comprising with claim 8, a product comprised of all-natural components, whereas at least a substantial amount of the solute portion constituents is used for therapeutic respiratory effects.

16. Comprising with claim 8, the present invention allows product substances to be transferred along the nasopharyngeal factory cells human airway epithelial cells, bypassing the whole body, and entering the nasal tract and lungs
